(19) United States
(12) Patent Application Publication
(10) Pub. No.: US 2021/0106232 A1
(43) Pub. Date: Apr. 15, 2021

(54) NON-CONTACT PHYSIOLOGICAL SIGNAL MEASURING DEVICE

(71) Applicant: FACEHEART INC., Hsinchu City (TW)

(72) Inventors: Tsuey-Huey SHIUE, Hsinchu City (TW); Kuan-Hung CHEN, Hsinchu City (TW); Chih-Wei LIU, Taipei City (TW); Po-Wei HUANG, Hsinchu City (TW); Bing-Jhang WU, Chiayi City (TW); Wen-Chung CHEN, Taoyuan City (TW)

(21) Appl. No.: 16/596,757

(22) Filed: Oct. 9, 2019

(51) Int. Cl. A61B 5/00; A61B 5/0205; A61B 5/024; A61B 5/01; A61B 5/1455

(52) U.S. Cl. CPC A61B 5/0077; A61B 5/0205; A61B 5/02405; A61B 2562/0233; A61B 5/14552; A61B 5/72; A61B 5/0022; A61B 5/01

(57) ABSTRACT

Disclosures of the present invention describe a non-contact physiological signal measuring device comprising a light sensing unit and a signal processing module, wherein the light sensing unit is adopted for collecting a scattered light from a surface of a sensing portion of a subject. After receiving the scattered light by a signal receiving unit of the signal processing module, a signal processing unit can subsequently obtain physiological characteristic(s) after applying signal process to a physiological signal with respect to the scattered light. Particularly, this novel non-contact physiological signal measuring device does not include any one camera or image capturing unit, such that the subject is able to receive a physiological signal measurement in the case of having well a personal privacy protection as well as preventing the subject's skin from being hurt.

### BACKGROUND OF THE INVENTION

### 1. Field of the Invention

The present invention relates to the technology field of physiological signal detection, and more particularly to a non-contact physiological signal measuring device that is able to complete a physiological signal measurement on a subject's skin in the case of having a well personal privacy protection as well as preventing the subject's skin from being hurt. Moreover, this non-contact physiological signal measuring device also has the advantage of having simple framework.

### 2. Description of the Prior Art

It is well known that, blood oxygen saturation and heart rate are two of multi physiological characteristics that are adopted for being as important health parameters. Recently, photoplethysmography (PPG), an optical measurement technique, has been widely used to measure a physiological signal from an individual, thereby extracting at least one physiological characteristic of the individual from the measured physiological signal. For example, Taiwan patent No. 1592138 discloses a wearable blood-pressure measuring apparatus having photoplenthysmography sensor units. After a user wears the wearable blood-pressure measuring apparatus on his wrist, the wearable blood-pressure measuring apparatus can be operated to achieve a physiological signal measurement on the wrist skin of the user. During the physiological signal measurement, the wearable blood-pressure measuring apparatus firstly emits a detecting light to the wrist skin of the user, so as to subsequently receive a reflection light radiated from the wrist skin by a light receiving unit thereof, thereby generating a PPG signal through successively recording variations in the amplitude of the reflection light. On the other hand, U.S. patent publication No. 2017/0340217A1 discloses a physiological detection device, which is actually a fingertip pulse oximeter. When using the physiological detection device to complete a physiological signal measurement of a subject, the subject is required to put his index finger into a finger receiving space of the fingertip pulse oximeter, such that a detecting light is generated and then emits to one surface of the index finger. Consequently, the fingertip pulse oximeter receives a transmitted light from another surface of the index finger, so as to eventually produce corresponding PPG signal after successively recording variations in the amplitude of the transmitted light.

From above descriptions, it is understood that photoplethysmography (PPG) method has applied to carry out a reflection-type contact physiological signal measuring device and/or a transmission-type contact physiological signal measuring device. However, user feedback has indicated that the conventional contact physiological signal measuring device shows some drawbacks in practical use. For example, the contact physiological signal measuring device commonly causes the skin allergy of the users who have sensitive skin. In view of that, China patent No. CN102973253B discloses a system for monitoring human physiological indexes by using visual information, which is a non-contact physiological signal measuring device. When the non-contact physiological signal measuring device is operated to execute a physiological signal measurement, a camera is firstly adopted for continuously capturing a subject image from a subject. Next, an image processing unit subsequently identifies the subject's face after completing a complex image processing of the subject image, so as to define and select a region of interest (ROI) from the subject's face. As a result, after splitting a red channel signal, a green channel signal and a blue channel signal from the selected ROI image frame(s), the R/G/B signals are conditioned in preparation for data analysis for the gathering of physiological characteristics.

The forgoing technology way also called imaging photoplethysmography (iPPG) or remote photoplethysmography (rPPG). Engineers skilled in use or application of the rPPG certainly know that, the above-mentioned image processing unit integrated in the non-contact physiological signal measuring device must be an image processor chip with high-speed computing ability, causing the manufacturing cost of the non-contact physiological signal measuring device fails to be effectively lowered. In addition, despite the fact that there is a high performance image processor integrated in the non-contact physiological signal measuring device using rPPG method, the non-contact physiological signal measuring device still needs a considerable time for finishing a huge computation, in order to extract physiological characteristics from the image of subject's face. The most important thing is that, resulted from the fact that there are large amounts of image data stored in a storage unit of the non-contact physiological signal measuring device during the physiological signal measurement using rPPG method, the subject therefore worries about lack of personal privacy.

As such, above descriptions have stated that the conventional contact physiological signal measuring device using PPG method includes advantages of simple framework and low cost, however, user feedback has still indicated that the contact physiological signal measuring device commonly causes the skin allergy of the users having sensitive skin. On the other hand, although the conventional non-contact physiological signal measuring device using rPPG method is able to achieve a physiological signal measurement of a subject by a non-contact way, the manufacturing cost of the non-contact physiological signal measuring device fails to be effectively lowered due to having a high performance image processor. Moreover, the subject often worries about lack of personal privacy when using the non-contact physiological signal measuring device.

In view of the fact that two principal types of physiological signal measuring devices both exhibit drawbacks in practical use, inventors of the present application have made great efforts to make inventive research and eventually provided a non-contact physiological signal measuring device.

### SUMMARY OF THE INVENTION

The primary objective of the present invention is to disclose a particularly-designed non-contact physiological signal measuring device, which not only shows the feature of measuring a physiological signal from a subject by a non-contact way that is exhibited by the conventional non-contact physiological signal measuring device using rPPG method, but also has the advantages of simple framework and low manufacturing cost that are the same as that of the conventional contact physiological signal measuring device using PPG method. In addition, this novel non-contact physiological signal measuring device does not include any one camera or image capturing unit, such that the subject is able to receive a physiological signal measurement in the case of having a well personal privacy protection as well as preventing the subject's skin from being hurt.

For achieving the primary objective of the present invention, the present invention provides an embodiment of the non-contact physiological signal measuring device, comprising:
a light sensing unit, being adopted for facing a sensing portion of a subject, thereby collecting a scattered light from a surface of the sensing portion by a non-contact way; and
a signal processing module, comprising:
   a signal processing unit;
   a control unit, being coupled to the signal processing unit and the light sensing unit, so as to control the light sensing unit to collect the scattered light; and
   a signal receiving unit, being coupled to the light sensing unit and the signal processing unit, and being configured for receiving the scattered light so as to transmit a physiological signal with respect to the scattered light to the signal processing unit;
wherein after receiving the physiological signal, the signal processing unit applying at least one signal process to the physiological signal, thereby obtaining at least one physiological information.

In the embodiment of the non-contact physiological signal measuring device, the physiological information comprises at least one physiological characteristic that is selected from the group consisting of blood volume, heart rate (HR), respiratory rate (RR), blood oxygen saturation, blood pressure, blood vessel viscosity, venous function, venous reflux, ankle pressure, genital response, and cardiac output.

In one practicable embodiment, the non-contact physiological signal measuring device further comprises:
a data outputting unit, being coupled to the signal processing unit, such that the signal processing unit is able to output the at least one physiological information through the data outputting unit;
wherein the data outputting unit is selected from the group consisting of display device, loudspeaker, wired transmission interface, and wireless transmission interface.

In one practicable embodiment, the non-contact physiological signal measuring device further comprises:
a condensing lens, being disposed between the light sensing unit and the scattered light, so as to focus the scattered light onto the light sensing unit;
wherein the scattered light is a mono-wavelength light or a multi-wavelength light.

In the embodiment of the non-contact physiological signal measuring device, the scattered light is produced at the surface of the sensing portion when the object is exposed to an ambient light, wherein the ambient light is a natural light or an artificial light provided by an external light source.

In one practicable embodiment, the non-contact physiological signal measuring device further comprises:
a light emitting unit for radiating a detecting light to the surface of the sensing portion, so as to make the scattered light be produced at the surface of the sensing portion;
wherein the light emitting unit comprises at least one light emitting component, and the light emitting component being selected from the group consisting of light emitting diode (LED), organic light-emitting diode (OLED) and vertical resonant cavity LED.

In one practicable embodiment, the non-contact physiological signal measuring device further comprises:
a driver unit, being coupled to the control unit, and being configured for driving the light emitting unit to radiate the detecting light.

In the embodiment of the non-contact physiological signal measuring device, the light sensing unit is selected from the group consisting of single point photo sensor, matrix photo sensor, one-channel image sensor and multi-channel image sensor.

In the embodiment of the non-contact physiological signal measuring device, the light sensing unit has an infrared (IR) sensor, such that the non-contact physiological signal measuring device has a function of body temperature measurement.

In one practicable embodiment, the non-contact physiological signal measuring device having the function of body temperature measurement is integrated in an optical-type body temperature measurement device.

In one practicable embodiment, the non-contact physiological signal measuring device further comprises:
a sensing region labeling unit, being coupled to the control unit, so as to label a mark on the surface of the sensing portion for pointing out a sensing region;
wherein the mark is selected from the group consisting of light spot, pattern, symbol, and text.

In one practicable embodiment, the non-contact physiological signal measuring device further comprises:
a living object determining unit, being coupled to the signal processing unit and/or the signal receiving unit, and being configured for applying at least one signal analyzing process to the physiological signal, so as to determine whether at least one physiological characteristic of living object is carried by the physiological signal or not, thereby recognizing the subject as one living object or one non-living object.

In the embodiment of the non-contact physiological signal measuring device, the physiological characteristic of living object is selected from the group consisting of frequency-domain physiological characteristic and time-domain physiological characteristic. In which, the frequency-domain physiological characteristic is a periodic heartbeat, and the time-domain physiological characteristic is a specific waveform feature of living object that is carried by the physiological signal.

In one practicable embodiment, the non-contact physiological signal measuring device, further comprises:
an alarming unit, being coupled to the living object determining unit, and being configured for showing a warning information in case of the subject being recognized as one non-living object by the living object determining unit.

### BRIEF DESCRIPTION OF THE DRAWINGS

The invention as well as a preferred mode of use and advantages thereof will be best understood by referring to the following detailed description of an illustrative embodiment in conjunction with the accompanying drawings, wherein:

FIG. 1 shows a first schematic stereo diagram of a first embodiment of a non-contact physiological signal measuring device according to the present invention;

FIG. 2 shows a function block diagram of the first embodiment of the non-contact physiological signal measuring device;

FIG. 3 shows a second schematic stereo diagram of the first embodiment of the non-contact physiological signal measuring device;

FIG. 4 shows a schematic stereo diagram of a second embodiment of the non-contact physiological signal measuring device according to the present invention;

FIG. 5 shows a function block diagram of the second embodiment of the non-contact physiological signal measuring device;

FIG. 6 shows a function block diagram of a third embodiment of the non-contact physiological signal measuring device;

FIG. 7 shows a function block diagram of a fourth embodiment of the non-contact physiological signal measuring device; and

FIG. 8 shows a function block diagram of a fifth embodiment of the non-contact physiological signal measuring device.

### DETAILED DESCRIPTION OF THE PREFERRED EMBODIMENTS

To more clearly describe a non-contact physiological signal measuring device disclosed by the present invention, embodiments of the present invention will be described in detail with reference to the attached drawings hereinafter.

### First Embodiment

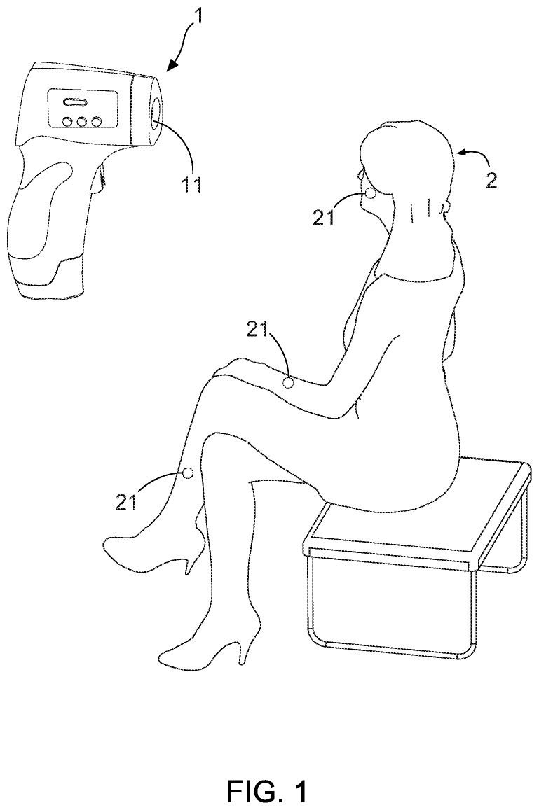

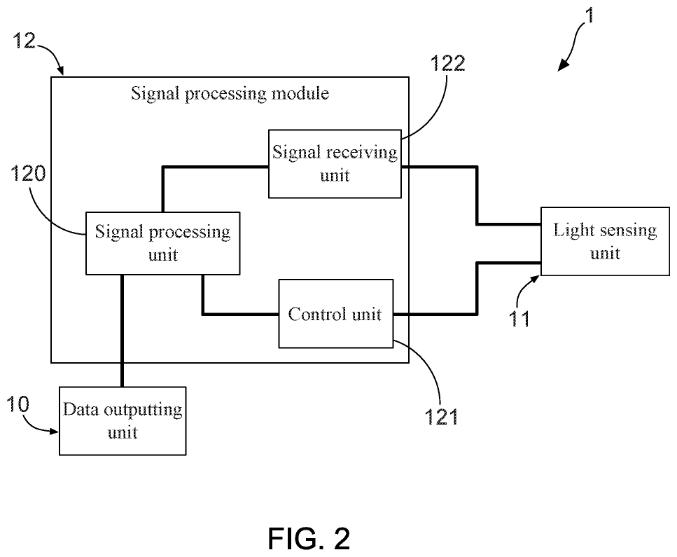

With reference to FIG. 1, there is shown a first schematic stereo diagram of a first embodiment of a non-contact physiological signal measuring device according to the present invention. Moreover, FIG. 2 shows a function block diagram of the first embodiment of the non-contact physiological signal measuring device. In first embodiment, particularly speaking, the non-contact physiological signal measuring device 1 shown in FIG. 1 and FIG. 2 has a simplest framework, and merely comprises a light sensing unit 11 and a signal processing module 12. According to the design of the present invention, the light sensing unit 11 is adopted for facing a sensing portion 21 of a subject 2, so as to collect a scattered light from a surface of the sensing portion 21 by a non-contact way. For example, FIG. 1 depicts that the subject 2 is a human, and the sensing portion 21 can be face, hand, or other human body portion that has surface skin.

When the subject 2 is exposed to an ambient light, the scattered light is produced at the surface of the sensing portion 21. It is worth noting that, since the ambient light can be a natural light or an artificial light provided by an external light source, the scattered light can therefore be a mono-wavelength light or a multi-wavelength light. As such, the light sensing unit 11 would not have a limited embodiment in the present invention. According to the scattered light may be a mono-wavelength light or a multi-wavelength light, the light sensing unit 11 is selected from the group consisting of single point photo sensor, matrix photo sensor, one-channel image sensor and multi-channel image sensor.

As described in more detail below, the signal processing module 12 comprises a signal processing unit 120, a control unit 121 and a signal receiving unit 122, wherein the control unit 121 is coupled to the signal processing unit 120 and the light sensing unit 11, thereby controlling the light sensing unit 11 to collect the scattered light. On the other hand, the signal receiving unit 122 is coupled to the light sensing unit 11 and the signal processing unit 120, and is particularly configured for receiving the scattered light so as to transmit a physiological signal with respect to the scattered light to the signal processing unit 120. Consequently, after receiving the physiological signal through the signal receiving unit 122, the signal processing unit 120 applies at least one signal process to the physiological signal, thereby obtaining physiological information. In general, the physiological information comprises at least one physiological characteristic that is selected from the group consisting of blood volume, heart rate (HR), respiratory rate (RR), blood oxygen saturation, blood pressure, blood vessel viscosity, venous function, venous reflux, ankle pressure, genital response, and cardiac output.

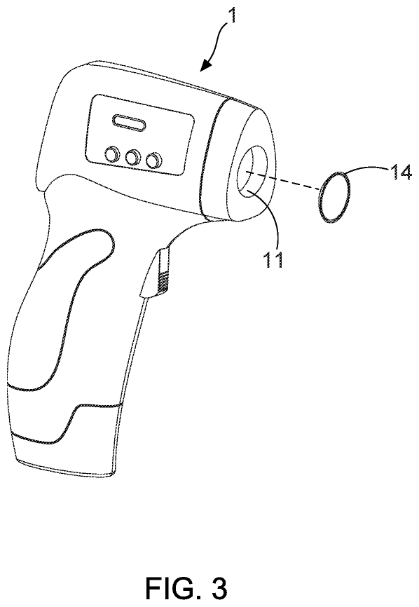

FIG. 2 also depicts that the non-contact physiological signal measuring device 1 comprises a data outputting unit 10, which is coupled to the signal processing unit 120, such that the signal processing unit 120 is able to output the at least one physiological information through the data outputting unit 10. In the present invention, the data outputting unit 11 would not have a limited embodiment. For instance, the data outputting unit 11 can be a display device, a loudspeaker, a wired transmission interface, or a wireless transmission interface. Please further refer to FIG. 3, which illustrate shows a second schematic stereo diagram of the first embodiment of the non-contact physiological signal measuring device. From FIG. 3, it is found that a condensing lens 14 is disposed between the light sensing unit 11 and the scattered light, and is used for focusing the scattered light onto the light sensing unit 11.

In addition, after detailedly observing the FIG. 1 and FIG. 3, it is certainly aware that the non-contact physiological signal measuring device 1 of the present invention is presented by a form of forehead thermometer. In one practicable embodiment, the non-contact physiological signal measuring device can further have a function of body temperature measurement as long as the light sensing unit 11 is designed to include an infrared (IR) sensor. As such, the non-contact physiological signal measuring device having the function of body temperature measurement can be further integrated in an optical-type body temperature measurement device, such as forehead thermometer or ear thermometer. Otherwise, the non-contact physiological signal measuring device 1 can directly be an optical-type body temperature measurement device having a specific function of physiological signal measurement.

### Second Embodiment

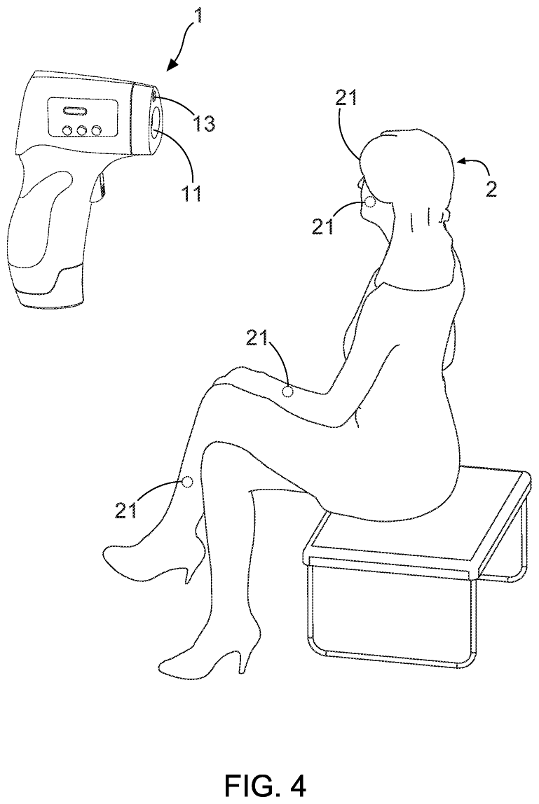

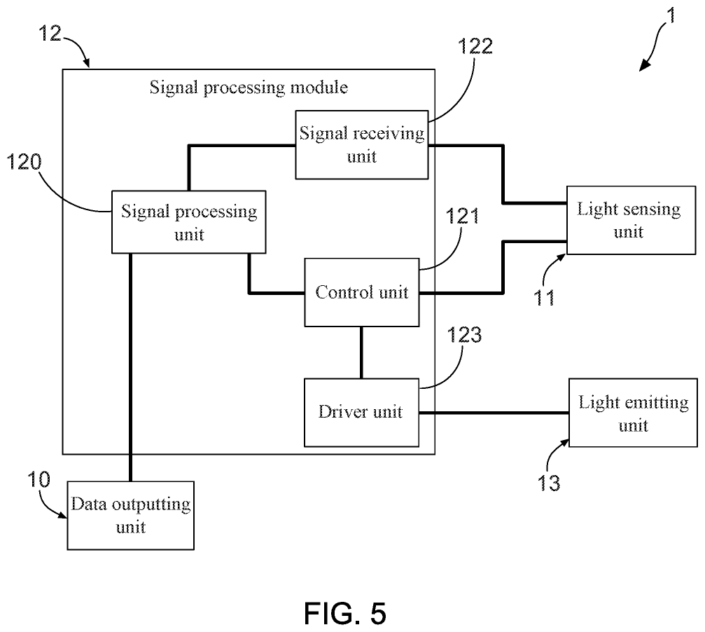

With reference to FIG. 4, there is shown a schematic stereo diagram of a second embodiment of the non-contact physiological signal measuring device according to the present invention. Moreover, FIG. 5 shows a function block diagram of the second embodiment of the non-contact physiological signal measuring device. After comparing FIG. 2 with FIG. 3, it is understood that the second embodiment of the non-contact physiological signal measuring device 1 further comprises a light emitting unit 13. In addition, in the second embodiment, the signal processing module 12 is designed to further comprises a driver unit 123, which is coupled to the control unit 121, and is configured for driving the light emitting unit 13 to radiate an artificial light for being as the detecting light, wherein the artificial light is a mono-wavelength light or a multi-wavelength light. As described in more detail below, the light emitting unit 13 comprises at least one light emitting component, and the light emitting component being selected from the group consisting of light emitting diode (LED), organic light-emitting diode (OLED) and vertical resonant cavity LED. It needs to further explain that, the forgoing LED can be a monochromatic light LED or a polychromatic light LED capable of emitting a green light (400-600 nm), a red light (600-800 nm) and an infrared (IR) light (800-1000 nm). Similarly, the forgoing OLED can be a monochromatic light OLED or a polychromatic light OLED capable of emitting a green light, a red light and an infrared (IR) light.

Briefly speaking, the first embodiment of the non-contact physiological signal measuring device 1 is designed to complete a physiological signal measurement of a subject 2 in the case of the subject 2 being exposed to a natural light or the non-contact physiological signal measuring device 1 does not include a light emitting unit (or light source). On the contrary, in the second embodiment, the non-contact physiological signal measuring device 1 is able to firstly use the light emitting unit 13 to radiate the detecting light so as to produce scattered light at the surface of one sensing portion 21 of the subject 2, thereby achieve a physiological signal measurement of the subject 2 by a non-contact way.

### Third Embodiment

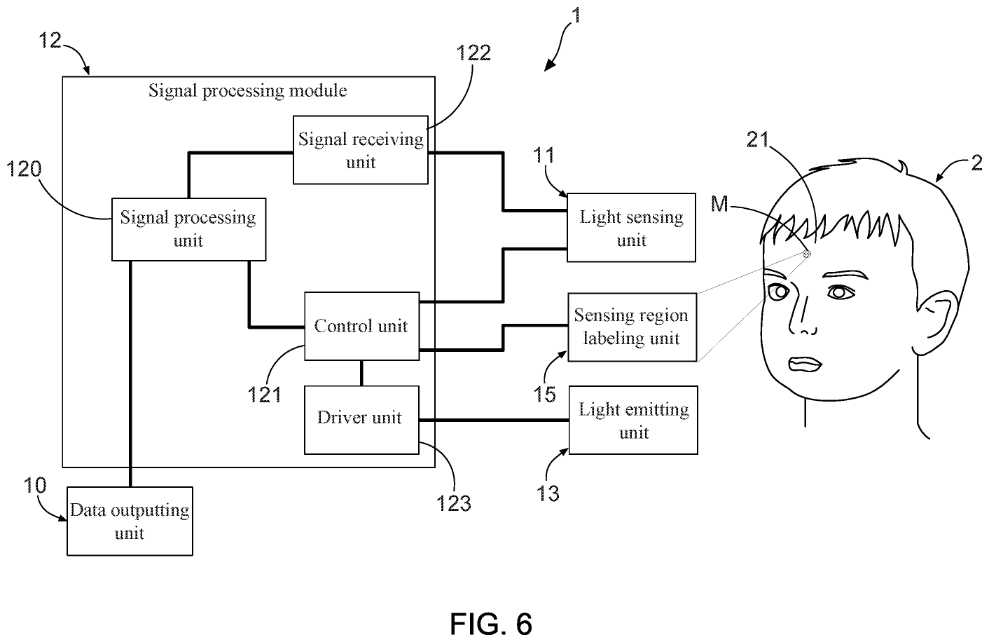

FIG. 6 shows a function block diagram of a third embodiment of the non-contact physiological signal measuring device. After comparing FIG. 5 with FIG. 6, it certainly knows that the third embodiment of the non-contact physiological signal measuring device 1 further comprises a sensing region labeling unit 15, which is coupled to the control unit 121, so as to label a mark on the surface of the sensing portion 21 for pointing out a sensing region M. For example, to label a light spot, a pattern, a symbol, or a text on the surface of the sensing portion 21. It needs further explain that, the sensing region labeling unit 15 is helpful in enhancement of the measurement accuracy of the non-contact physiological signal measuring device 1. As described in more detail below, in the case of the non-contact physiological signal measuring device 1 not including the light emitting unit 13 or the detecting light is an IR light, a user of the non-contact physiological signal measuring device 1 fails to exactly determine where is the correct sensing portion 21 that the light sensing unit 11 is facing to. Especially, if the sensing portion 21 is the forehead of the subject 2, there is a high possibility for the user to make the light sensing unit 11 face to the subject's forehead that is covered by hair. In such case, a physiological signal with respect to the scattered light cannot completely reflect a real physiological condition of the subject 2. From above descriptions, it is aware that the user is capable of making the light sensing unit 11 face to the subject's forehead correctly as long as the sensing region labeling unit 15 points out a sensing region M on the forehead (i.e., sensing portion 21) of the subject 2. As a result, the physiological signal with respect to the scattered light can completely reflect the real physiological condition of the subject 2, such that the measurement accuracy of the non-contact physiological signal measuring device 1 is therefore enhanced.

### Fourth Embodiment

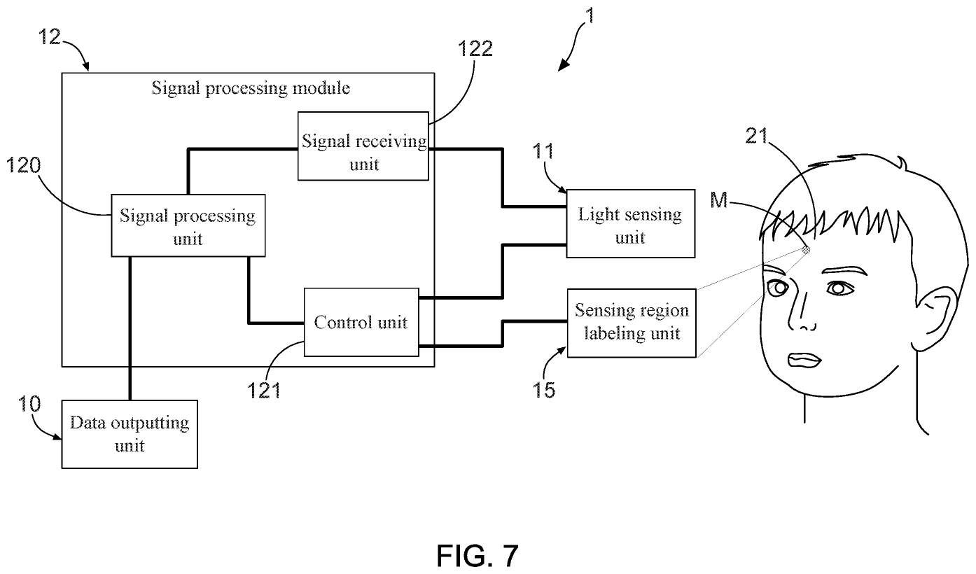

FIG. 7 shows a function block diagram of a fourth embodiment of the non-contact physiological signal measuring device. After comparing FIG. 2 with FIG. 7, it is found that the fourth embodiment is easily established by adding the sensing region labeling unit 15 into the framework of the first embodiment. When using the fourth embodiment, user is able to make the light sensing unit 11 face to the subject's forehead correctly by using the sensing region labeling unit 15 to point out a sensing region M on the sensing portion 21 of the subject 2. Therefore, the physiological signal with respect to the scattered light can completely reflect the real physiological condition of the subject 2, such that the measurement accuracy of the non-contact physiological signal measuring device 1 is eventually enhanced.

### Fifth Embodiment

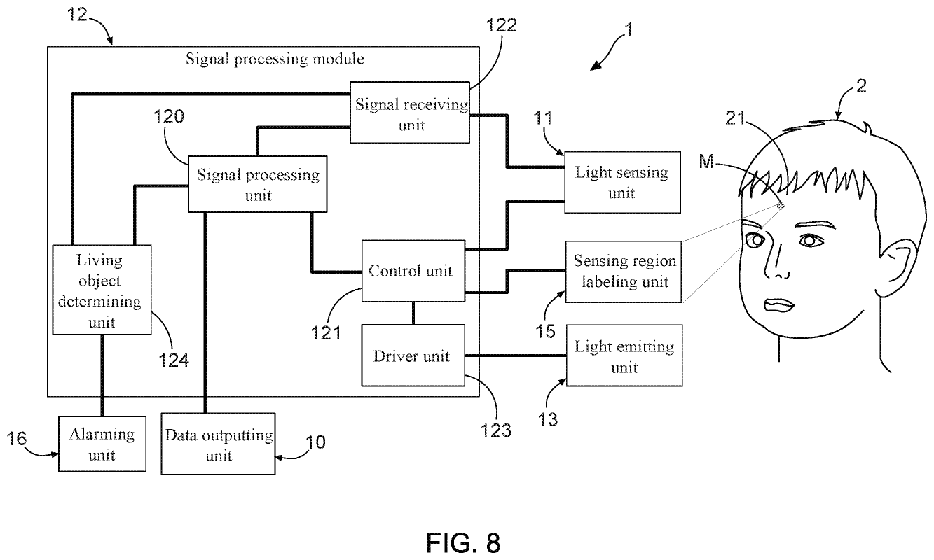

FIG. 8 illustrates a function block diagram of a fifth embodiment of the non-contact physiological signal measuring device. After comparing FIG. 6 with FIG. 8, it certainly finds that the fifth embodiment of the non-contact physiological signal measuring device 1 further comprises a living object determining unit 124, which is provided in the signal processing module 12, and is coupled to the signal processing unit 120 and the signal receiving unit 122. In fifth embodiment, the living object determining unit 124 is configured for applying at least one signal analyzing process to the physiological signal, so as to determine whether at least one physiological characteristic of living object is carried by the physiological signal or not, thereby recognizing the subject 2 as one living object or one non-living object. It is worth explaining that, in one practicable embodiment, the living object determining unit 124 can be arranged to merely connect with the signal processing unit 120 or the signal receiving unit 122. In addition, the fifth embodiment of the non-contact physiological signal measuring device 1 also comprises an alarming unit 16 that is coupled to the living object determining unit 124.

As described in more detail below, through the signal receiving unit 122, the living object determining unit 124 is able to receive a physiological signal with respect to the scattered light, i.e., a photoplethysmography (PPG) signal. A few of research reports and/or literatures have indicated that, the PPG signal measured from a living object commonly carries with some special frequency-domain physiological characteristics. Therefore, after applying at least one signal analyzing process to the PPG signal, the living object determining unit 124 can determine whether at least one physiological characteristic of living object is carried by the PPG signal or not, thereby recognizing the subject 2 as one living object or one non-living object. The forgoing physiological characteristic is the frequency-domain physiological characteristic. In an exemplarily embodiment, the said time-domain physiological characteristic is a specific waveform feature of living object that is carried by the physiological signal. In general, before extracting the frequency-domain physiological characteristic from the PPG signal, the living object determining unit 124 must complete a time domain signal processing of the PPG signal, like a signal processing using Singular Spectrum Analysis (SSA) or a signal processing using Normalized Least Mean Square (NLMS).

On the other hand, after finishing a frequency domain signal processing of the PPG signal, at least one frequency-domain physiological characteristic can be extracted from the post-processed PPG signal by the living object determining unit 124. From example, the time domain signal processing is a signal processing using Fast Fourier Transform (FFT) or a signal processing using Short-Time Fourier Transform (STFT). In an exemplarily embodiment, the said frequency-domain physiological characteristic is a periodic heartbeat, such that the living object determining unit 124 can therefore recognize the subject 2 as one living object or one non-living object through the periodic heartbeat.

The living object determining unit 124 is helpful in calibration and/or enhancement of the measurement accuracy of the non-contact physiological signal measuring device 1. As described in more detail below, in the case of a user taking this non-contact physiological signal measuring device 1 and then making the light sensing unit 11 face a non-living object, the living object determining unit 124 will immediately know that the light sensing unit 11 is facing a non-living object. Accordingly, the alarming unit 16 coupled to the living object determining unit 124 is configured for showing a warning information in case of the subject 2 being recognized as one non-living object by the living object determining unit 124. The forgoing warning information can be presented by a form of light, sound, text, or image. In addition, the data outputting unit 10 can also be configured to send out the warning information.

The above description is made on embodiments of the non-contact physiological signal measuring device according to the present invention. However, the embodiments are not intended to limit scope of the present invention, and all equivalent implementations or alterations within the spirit of the present invention still fall within the scope of the present invention.

What is claimed is:

1. A non-contact physiological signal measuring device, comprising:
a light sensing unit 11, being adopted for facing a sensing portion 21 of a subject 2, thereby collecting a scattered light from a surface of the sensing portion 21 by a non-contact way; and
a signal processing module 12, comprising:
a signal processing unit 120;
a control unit 121, being coupled to the signal processing unit 120 and the light sensing unit 11, so as to control the light sensing unit 11 to collect the scattered light; and
a signal receiving unit 122, being coupled to the light sensing unit 11 and the signal processing unit 120, and being configured for receiving the scattered light so as to transmit a physiological signal with respect to the scattered light to the signal processing unit 120;
wherein after receiving the physiological signal, the signal processing unit 120 applying at least one signal process to the physiological signal, thereby obtaining at least one physiological information.

2. The non-contact physiological signal measuring device of claim 1, wherein the physiological information comprises at least one physiological characteristic that is selected from the group consisting of blood volume, heart rate (HR), respiratory rate (RR), blood oxygen saturation, blood pressure, blood vessel viscosity, venous function, venous reflux, ankle pressure, genital response, and cardiac output.

3. The non-contact physiological signal measuring device of claim 1, further comprising:
a data outputting unit 10, being coupled to the signal processing unit 120, such that the signal processing unit 120 is able to output the at least one physiological information through the data outputting unit 10.

4. The non-contact physiological signal measuring device of claim 3, wherein the data outputting unit 10 is selected from the group consisting of display device, loudspeaker, wired transmission interface, and wireless transmission interface.

5. The non-contact physiological signal measuring device of claim 1, further comprising a condensing lens 14 for being disposed between the light sensing unit 11 and the scattered light, so as to focus the scattered light onto the light sensing unit 11.

6. The non-contact physiological signal measuring device of claim 1, wherein the scattered light is produced at the surface of the sensing portion 21 when the subject 2 is exposed to an ambient light.

7. The non-contact physiological signal measuring device of claim 6, wherein the ambient light is a natural light or an artificial light provided by an external light source.

8. The non-contact physiological signal measuring device of claim 1, further comprising a light emitting unit 13 for radiating a detecting light to the surface of the sensing portion 21, so as to make the scattered light be produced at the surface of the sensing portion 21.

9. The non-contact physiological signal measuring device of claim 8, wherein the light emitting unit 13 comprises at least one light emitting component, and the light emitting component being selected from the group consisting of light emitting diode (LED), organic light-emitting diode (OLED) and vertical resonant cavity LED.

10. The non-contact physiological signal measuring device of claim 9, wherein the signal processing module 12 further comprises a driver unit 123 that is coupled to the control unit 121, and being configured for driving the light emitting unit 13 to radiate the detecting light.

11. The non-contact physiological signal measuring device of claim 1, wherein the scattered light is a mono-wavelength light or a multi-wavelength light.

12. The non-contact physiological signal measuring device of claim 1, wherein the light sensing unit 11 is selected from the group consisting of single point photo sensor, matrix photo sensor, one-channel image sensor and multi-channel image sensor.

13. The non-contact physiological signal measuring device of claim 1, wherein the light sensing unit 11 has an infrared (IR) sensor, such that the non-contact physiological signal measuring device has a function of body temperature measurement.

14. The non-contact physiological signal measuring device of claim 13, wherein the non-contact physiological signal measuring device having the function of body temperature measurement is integrated in an optical-type body temperature measurement device.

15. The non-contact physiological signal measuring device of claim 1, further comprising:
a sensing region labeling unit 15, being coupled to the control unit 121, so as to label a mark on the surface of the sensing portion 21 for pointing out a sensing region M.

16. The non-contact physiological signal measuring device of claim 1, further comprising:
a living object determining unit 124, being coupled to the signal processing unit 120 and/or the signal receiving unit 122, and being configured for applying at least one signal analyzing process to the physiological signal, so as to determine whether at least one physiological characteristic of living object is carried by the physiological signal or not, thereby recognizing the subject 2 as one living object or one non-living object.

17. The non-contact physiological signal measuring device of claim 15, wherein the mark is selected from the group consisting of light spot, pattern, symbol, and text.

18. The non-contact physiological signal measuring device of claim 16, wherein the physiological characteristic of living object is selected from the group consisting of frequency-domain physiological characteristic and time-domain physiological characteristic.

19. The non-contact physiological signal measuring device of claim 16, further comprising:
an alarming unit 16, being coupled to the living object determining unit 124, and being configured for showing a warning information in case of the subject 2 being recognized as one non-living object by the living object determining unit 124.

20. The non-contact physiological signal measuring device of claim 18, wherein the frequency-domain physiological characteristic is a periodic heartbeat, and the time-domain physiological characteristic being a specific waveform feature of living object that is carried by the physiological signal.
